(19) United States
(12) Patent Application Publication
(10) Pub. No.: US 2007/0021508 A1
(43) Pub. Date: Jan. 25, 2007

(54) Method for treatment of Helicobacter pylori infection and/or an associated disease

(75) Inventors: Rong-Lang Yen, Yongkang City (TW); Chung-Yi Lo, Tainan (TW)

Correspondence Address: ANDREWS KURTH LLP, 1350 I STREET, N.W., SUITE 1100, WASHINGTON, DC, 20005, US

(73) Assignee: ANTROMED INC

(21) Appl. No.: 11/183,892

(22) Filed: Jul. 19, 2005

(51) Int. Cl. A61K 31/192

(52) U.S. Cl. 514/569; 514/570

(57) ABSTRACT

The present invention relates to a method for treating a Helicobacter pylori (IP) infection and/or an associated disease in an individual, comprising administering to the individual a therapeutically effective amount of a pharmaceutical composition containing a short-chain fatty acid or a pharmaceutically acceptable salt thereof, alone or in combination with an anti-HP agent, and a pharmaceutically acceptable carrier, wherein the short-chain fatty acid has the formula:

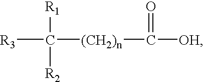

in which R₁ and R₂, independently, are C₁-C₈alkyl or H, R₃ is aryl or heteroaryl, and n is 0, 1, 2, 3, 4, 5 or 6. The present invention also relates to a method for treatment of Helicobacter pylori (HP) infection or disease associated with HP infection by administering a therapeutically effective amount of an HDAC inhibitor or a pharmaceutically acceptable salt thereof, alone or in combination with an anti-HP agent, and a pharmaceutically acceptable carrier.

### BACKGROUND OF THE INVENTION

1. Field of Invention

The present invention relates to a method for the treatment of Helicobacterpylori (HP) infection and/or an associated disease, particularly including chronic atrophic gastritis, gastric or duodenal ulcer, gastric adenocarcinoma, gastroesophageal reflux, peptic esophagitis, squamous cell esophageal cancer, mucosa-associated lymphoid tissue lymphomas (MALTomas), iron deficiency anemia, skin disease, coronary artery disease, idiopathic thrombocytopenic purpura and rheumatological diseases.

2. Description of the Related Art

Helicobacterpylori (HP) is a micro-aerophilic, spiral-shaped gram-negative bacillus responsible for one of the most common infections found in humans. First identified as Campylobacter pylori by Warren and Marshall, the bacillus was later renamed Helicobacter pylori and recognized to be associated with gastritis and duodenal ulcers. HP may be detected in approximately 90% of individuals with peptic ulcer diseases (PUD). Several international surveys indicated that at least half of all people are infected with HP, and about 15% of the infected individuals may develop PUD.

HP is motile because of the flagella. HP also produces important disease-inducing factors, including urease, vacuolating cytotoxin Vac-A, Cag-A protein, catalase, and lipopolysaccharide (LPS). Urease converts urea to ammonium and bicarbonate, thereby neutralizing gastric acid and providing protection until HP reaches the submucosal layer (microenvironment with neutral pH). The flagella help HP pass from the acidic gastric lumen to inhabit the gastric mucus. The Cag-A and Vac-A proteins serve to induce pronounced inflammation and increased propensity to cause disease. Catalase helps HP survive in the host by preventing the formation of free radicals from hydrogen peroxide in neutrophils. The LPS outer membrane of HP bacteria is a less potent inducer of the host immune response and enhances the ability of HP to colonize the stomach. HP also produces and releases several bioactive factors that may affect the stomach's parietal cells to secret more hydrochloric acid. In conclusion, HP colonizes the stomach, induces inflammatory cytokines, causes gastric inflammation (with polymorphonuclear and mononuclear cell infiltration), and ulcer formation.

PUD is a common disorder of gastric or duodenal mucosa tissues, with mucosal break, 3 mm or greater in size with depth. The major causes are HP infection and nonsteroidal anti-inflammatory drugs (NSAIDs). However, HP strains differ in their potential to cause diseases. The hypothesized mechanisms of duodenal ulcers are hypergastrinemia and delivery of high gastric acid levels to the duodenum.

HP also seems to be involved in the pathogenesis of other diseases, such as chronic atrophic gastritis, adenocarcinoma of the body or antrum of the stomach, gastroesophageal reflux disease, peptic esophagitis, squamous cell esophageal cancer, mucosa-associated lymphoid tissue lymphomas (MALTomas), iron deficiency anemia, skin disease, coronary artery inflammation, idiopathic thrombocytopenic purpura (due to anti-CagA antibodies that cross-react with platelet antigens) and rheumatological diseases. One pathological model for tumor formation is the stepwise progression of HP infection to chronic gastritis, atrophic gastritis, intestinal metaplasia and gastric cancer. Some co-factors may also play key roles in determining the progression. Several large multicenter studies suggested that long-term remissions in 70% to 80% of low-grade MAlToma cases can be induced by eradication of HP. However, the prognosis is usually poor for patients who later develop squamous cell esophageal cancer or gastric adenocarcinoma. As chronic atrophic gastritis is a precancerous lesion for gastric cancer and the eradication of HP infection can halt chronic gastritis, eradication of HP may prevent gastric cancer. Therefore, eradication of HP infection is recommended for patients with stomach corpus atrophy.

The US Food and Drug Administration (FDA) has approved some regimens for the treatment of HP infection in patients with gastric or duodenal ulcers. Some of the approved regimens are omeprazole, amoxicillin and clarithromycin (OAC); bismuth subsalicylate, metronidazole and tetracycline (BMT); and lansoprazole, amoxicillin and clarithromycin (LAC). Some investigators have reported that eradication of HP by these regimens resulted in the cure of chronic gastritis, decreased recurrence of PUD and decreased incidence of gastric cancer or MALTomas. However, antibiotics have resistance and toxicity problems nowadays. Nitroimidazole or clarithromycin resistance is becoming an important problem. HP isolates that have been found resistant to metronidazole vary from 20% in New Zealand to 80-90 % in China, Zaire and Bangladesh. Metronidazole resistance mutations also confer cross-resistance to other nitroimidazoles. Resistance in HP to nitroimidazoles appears unlikely to decrease over time. Besides, clarithromycin resistance has been increasingly reported with rates ranging from 10 to 40 %. Either primary or acquired resistance to clarithromycin jeopardizes the success of the first-line regimen, because the cure rate varied from 20-50% (wherein the HP isolates were clarithromycin-resistant) to 64-98% (for susceptible isolates).

Furthermore, all the eradication regimens have a high incidence of side-effects (for example, nausea, skin rash, vomiting, diarrhea and metallic taste). If skin rash, vomiting or diarrhea occurs, the treatment course is discontinued. A need exists for more effective and less toxic regimens to eradicate HP infection and/or an associated disease.

An approach to selectively eradicate HP infection or its associated diseases without unacceptable toxicity is needed. Besides, HP infection is associated with several malignancies including gastric adenocarcinoma, squamous cell esophageal cancer and MALTomas. Thus, a need still exists to eradicate HP infection locally, to decrease the toxicity of conventional anti-HP regimens, to promote healing of gastrointestinal ulcers, and to inhibit the late sequela of HP infection (tumor cell proliferation) in treating HP infection and/or an associated disease.

### SUMMARY OF THE INVENTION

The present invention is to provide a method for treatment of Helicobacter pylori infection and/or an associated disease. The method according to the present invention comprises administering to the individual a therapeutically effective amount of a pharmaceutical composition containing a short-chain fatty acid or a pharmaceutically acceptable salt thereof, alone or in combination with one or more anti-HP agents, and a pharmaceutically acceptable carrier, wherein the short-chain fatty acid has the formula:

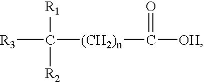

in which R₁ and R₂, independently, are C₁-C₈alkyl or H, R₃ is aryl or heteroaryl, and n is 0, 1, 2, 3, 4, 5 or 6.

Other advantages or features of the present invention will be demonstrated in the following description of several embodiments, and also from the appending claims.

### DETAILED DESCRIPTION OF THE INVENTION

The present invention is based on the unexpected discovery that certain short-chain fatty acids and in particular 2-phenylbutyrate (2-PB) and 4-phenylbutyrate (4-PB) strongly inhibit the growth of HP, either alone or in combination with anti-HP antibiotics or other regimens. Besides, anti-HP activity does not seem to be a general phenomenon for short-chain fatty acids with butyric backbone, or other histone deacetylase (HDAC) inhibitors. These compounds can be integrated into a pharmaceutical composition to treat HP infection and/or an associated disease in an individual by administering to the individual a therapeutically effective amount of a short-chain fatty acid or a pharmaceutically acceptable salt thereof, alone or in combination with a agent, and a pharmaceutically acceptable carrier. The diseases associated with HP infection include chronic atrophic gastritis, gastric or duodenal ulcers, gastric adenocarcinoma, gastroesophageal reflux, peptic esophagitis, squamous cell esophageal cancer, mucosa-associated lymphoid tissue lymphomas (MALTomas), iron deficiency anemia, skin disease, coronary artery disease, idiopathic thrombocytopenic purpura and rheumatological diseases. As chronic atrophic gastritis associated with HP infection is a precancerous lesion for gastric cancer and the eradication of HP infection can halt chronic gastritis, eradication of HP may prevent gastric cancer. Besides, the expected annual incidence of gastric cancer in patients with corpus atrophy with persistent HP infection was at least 5.8-fold higher than that for esophageal adenocarcinoma after the eradication of infection at all ages. Therefore, eradication of HP infection is highly recommended for HP-infected patients with stomach corpus atrophy.

Thus, this invention features a method for treating an individual with an HP infection and/or an associated disease. The short-chain fatty acids that may be employed according to the present invention can be purchased from commercial suppliers or synthesized by well-known methods, having the formula:

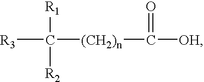

wherein R₁ and R₂, independently, are H or C₁-C₈alkyl; R₃ is aryl or heteroaryl; and n is 0, 1, 2, 3, 4, 5 or 6. A subset of the short-chain fatty acids that may be employed according to the present invention are preferred wherein R₃ is phenyl, n is 0, 1 or 2, and R₁ and R₂, independently, are H, methyl or ethyl. Two exemplary compounds are 2-phenylbutyrate (2-PB) and 4-phenylbutyrate (4-PB).

The terms “alkyl,” “aryl” and“heteroaryl” as referred to herein include both unsubstituted and substituted moieties.

The term “substituted” refers to one or more substituents, which may be the same or different, each in replace of a hydrogen atom. Examples of substituents include, but are not limited to, amino, cyano, halogen, hydroxyl, mercapto, C₁-C₈alkyl, C₁-C₈alkenyl, C₁-C8alkoxy, aryl, heteroaryl or heterocyclyl. Alkyl, alkenyl, alkoxy, aryl, heteroaryl and heterocyclyl are optionally substituted with C₁-C₈alkyl, halogen, amino, hydroxyl, mercapto or cyano.

The term “aryl” refers to a hydrocarbon ring system having at least one aromatic ring. Examples of aryl moieties include, but are not limited to, phenyl, naphthyl and pyrenyl.

The term “heteroaryl” refers to a hydrocarbon ring system having at least one aromatic ring that contains at least one heteroatom such as N, O or S. Examples of heteroaryl moieties include, but are not limited to carbozolyl and indolyl.

The term “short-chain fatty acids” herein refers to all the compounds of the formula 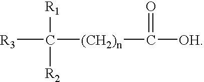
Salts or prodrugs of the short-chain fatty acids, if applicable, may be employed according to the present invention. The salts that may be employed according to the present invention can be formed between a cation and a negatively charged substituent (e.g., carboxylate) on a short-chain fatty acid compound. Suitable cations include, but not limited to, a calcium ion, a magnesium ion, a potassium ion, a sodium ion and an ammonium cation. Examples of prodrugs include esters or other pharmaceutically acceptable derivatives, which are capable of providing the short-chain fatty acids described above when administered to an individual.

The term “treatment” as used herein refers to administering a composition to an individual with the purposes of curing, improving or preventing HP infection, a disease associated with HP infection, its symptoms or the predisposition toward it.

Also within the scope of this invention is the use of the above-described compounds for the manufacture of a medicament for the treatment of a disease associated with HP infection.

An anti-HP agent in combination with the short-chain fatty acid according to the present invention is selected from the group consisting of proton pump inhibitors (omeprazole, lansoprazole), antibiotics (amoxicillin, tetracycline, doxycycline, minocycline, metronidazole, tinidazole, clarithromycin, roxithromycin), and cytoprotective bismuth subsalicylate. They are chosen to combine with said short-chain fatty acid to promote eradication of HP infection or its associated diseases. Besides, when the said short-chain fatty acid is combined with the above anti-HP agents, the amount of the anti-HP agent required to halt the growth of HP is lower compared to administering the anti-HP agent alone. Furthermore, when the anti-HP agent is combined with the short-chain fatty acid, the varieties of the anti-HP agent and the resistance of the anti-HP agent will be reduced. For example, amoxicillin is the least toxic among the group of anti-HP agents. When phenylbutyrate is combined with amoxicillin, the therapeutically effective dose of amoxicillin will be lowered, and the therapeutic toxicity will be much decreased. Besides, the administration of other more toxic anti-HP antibiotics might be reduced, and toxicity associated with these antibiotics will be reduced with reduced doses, or HP resistance to these agents might be decreased when the said short-chain fatty acid is combined with the above anti-HP agent.

Thus, the present invention provides a method for increasing therapeutic gain during the treatment of HP infection and/or an associated disease in an individual by simultaneously eradicating HP infection locally, decreasing toxicity and resistance to the anti-HP agents, promoting healing of mucosa ulcers, and inhibiting the late sequela of HP infection (tumor cell proliferation).

Phenylbutyrate (PB) is an aromatic short-chain fatty acid that can be purified from a biological sample (e.g., mammalian plasma or urine) or chemically synthesized. It belongs to a group of compounds called histone deacetylase (HDAC) inhibitors that cause inhibition of histone deacetylase, resulting in chromosomal histone hyperacetylation. The histone hyperacetylation in various gene loci results in up- or down-regulation of various genes in animal cells, and thereby causes induction of regrowth of non-malignant epithelial cells, cell differentiation and apoptotic cell death in tumor cells, and inhibition of tumor angiogenesis. Currently compounds of the HDAC inhibitors fall into seven structurally diverse classes, comprising: phenylbutyrate and valproic acid of the short-chain fatty acid class, trichostatin A of the hydroxamic acid class, depudecin of the epoxide class, trapoxin A of the cyclic tetrapeptide class, depsipeptide of the cyclic tetrapeptide class, the benzamide class and electrophilic ketone derivatives. PB has been approved by the U.S. Food and Drug Administration as an orphan drug for treating hyperammonemia caused by urea cycle disorder, and has also been tested in treatment of several diseases. In sickle cell anemia, PB stimulates transcription of the normal fetal hemoglobin gene to substitute for the mutated adult hemoglobin. In adrenoleukodystrophy, PB increases production of ALDRP to substitute for the mutated ABC transporter, thus preventing the accumulation of long-chain fatty acids. PB is also known to promote differentiation of cancer cells, as observed in prostate cancer cells and acute promyelocytic leukemia and is therefore being used in several clinical anti-cancer trials. PB was reported to be very safe during a 26-month follow-up in ornithine transcarbamylase-deficient patients. The patients were given a median dose of 352 mg/kg/day, and during that time, neither hyperammonemic episodes requiring hospitalization nor side effects related to therapy occurred. In animal studies, PB can even extend the lifespan and improve maintenance of vigor by enhancing the expression of genes that are involved in detoxification (for example, superoxide dismutase, glutathione S-transferase and cytochrome P450) or encoding chaperone proteins. Therefore, PB is a safe compound with the potential to promote healing of mucosa defect and inhibit tumor cell growth.

PB can be chemically synthesized or purchased from commercial suppliers. In a preferred embodiment of the present invention, PB has the ability to inhibit the growth of HP and increases the therapeutic gain for HP treatment regimens.

In a preferred embodiment of the present invention, some short-chain fatty acids (4-PB and 2-PB) were found to have much stronger ability to inhibit the growth of HP by creating a bigger clear zone in the HP lawn. Not all other phenylbutyrate-like compounds, including gama-aminobutyric acid (GABA), 2-aminobutyric acid (2-AB) and 2, 4-diaminobutyric acid (2, 4-DAB), have similar anti-HP activity.

This invention relates to a method for treatment of Helicobacter pylori (HP) infection or disease associated with HP infection by administering a therapeutically effective amount of a short-chain fatty acid or a pharmaceutically acceptable salt thereof, alone or in combination with an anti-HP agent, and a pharmaceutically acceptable carrier. The diseases associated with HP infection include chronic atrophic gastritis, gastric or duodenal ulcers, gastric adenocarcinoma, gastroesophageal reflux, peptic esophagitis, squamous cell esophageal cancer, mucosa-associated lymphoid tissue lymphomas (MALTomas), iron deficiency anemia, skin disease, coronary artery disease, idiopathic thrombocytopenic purpura and rheumatological diseases.

An effective amount of a short-chain fatty acid according to the present invention is the amount that, upon administration to an individual in need of treatment of a disease associated with HP infection, is required to confer a therapeutic effect on the individual. It may range from 20 mg/kg/day to 500 mg/kg/day. As recognized by those skilled in the art, the effective doses vary depending on the route of administration, excipient usage and the possibility of co-administration with other therapeutic regimens such as the use of other anti-ulcer or antibiotics. The recommended median dose of 4-PB for an ornithine transcarbamylase-deficient (urea cycle disorder) patient is 352 mg/kg/day, and neither hyperammonemic episodes requiring hospitalization nor side effects related to therapy were manifested during a 26-month follow-up.

Effective amounts and treatment regimens for any particular individual also depend on other factors, such as the activity of the specific compound administered, the age, sex, diet, body weight, health status, timing of administration, rate of excretion, the severity and course of the diseases and the patient's disposition to the diseases.

The short-chain fatty acids according to the present invention may be employed in the form of pharmaceutically acceptable salts. As such, they may be used as long as they do not adversely affect the safety concern or desired pharmaceutical effects of the compounds. The selection and production can be performed by those who are skilled in the art. Examples of pharmaceutically acceptable salts include alkali metal salts such as a potassium salt or a sodium salt, alkaline earth metal salts such as a calcium salt or a magnesium salt, salts with an organic base such as an ammonium salt or a salt with an organic base such as a triethylamine salt or an ethanolamine salt.

A pharmaceutically acceptable carrier may include water, a solvent, a preservative, a surfactant or a combination of the pharmaceutically acceptable carriers. Water, when present, can be in an amount of about 3 to about 98% by weight. Other than water, the pharmaceutically acceptable carrier can also contain a relatively volatile solvent such as a monohydric C₁-C₃ alkanol (e.g., ethyl alcohol) in an amount of about 1 to about 70% by weight and an emollient such as those in the form of silicone oils and synthetic esters in an amount of about 0.1 to about 30 % by weight. Anionic, nonionic or cationic surfactants may also be included in the pharmaceutically acceptable carrier. The concentration of total surfactants may be from 0.1 to 40% by weight.

The short-chain fatty acids of the present invention may be administered orally, intravenously, or intra-arterially. In the case of oral administration, they may be administered in the form of soft or hard capsules, tablets, powders, granules, solutions, suspensions or the like. Capsules may contain any standard pharmaceutically acceptable materials such as gelatin or cellulose. Tablets, on the other hand, may be formulated in accordance with conventional procedures by compressing mixtures of a short-chain fatty acid with a solid carrier and a lubricant. Examples of solid carriers include starch and sugar bentonite. In the case of non-oral administration, they may be administered in the form of an injection solution, a drip infusion formulation or liposome formations. The selection of the method for the delivery of these formulations and the vehicles, disintegrators or suspending agents can be readily made by those skilled in the art. The short-chain fatty acids of the present invention may contain a further substance having anti-acid or antibiotic activity or a pharmaceutically acceptable salt thereof, in addition to 4-PB or 2-PB, and a pharmaceutically acceptable carrier.

As recognized by those skilled in the art, the effective doses vary depending on the route of administration, excipient usage and the possibility of co-use with other therapeutic treatments such as the use of other anti-inflammatory or anti-tumor agents. Effective amounts and treatment regimens for any particular subject (e.g., mammalian, such as human, dog, or cat) will also depend upon a variety of other factors, including the activity of the specific compound employed, age, body weight, general health status, sex, diet, time of administration, rate of excretion, severity and course of the disease and the patient's disposition to the disease, but are usually from about 0.1 to about 50% by weight regardless of the manner of administration.

To understand the invention described herein more readily, the following examples are set forth. These examples are for illustrative purposes only and are not to be construed as limiting this invention in any manner.

All references cited herein are expressly incorporated by reference in their entirety.

### EXAMPLE 1

HP Strains

The HP strains used in the present studies are H. pylori ATCC43504 (HP 43504) and HP 238. HP 238 was isolated from a patient with MALToma in National Cheng Kung University Hospital, Tainan, Taiwan, and used as an antibiotic sensitive control. These strains were cultured under a microaerophilic condition (O₂ 5%, CO₂ 10%, N₂ 85%) in a 37° C. chamber. The minimum inhibitory concentration (MIC) against HP 43504 is 256 μg/ml for metronidazole, 0.12 μg/ml for amoxicillin, and 1 μg/ml for tetracycline. The MIC of metronidazole against the clinically isolated strain HP 238 is 0.25 μg/ml.

### EXAMPLE 2

Effect of Short-Chain Fatty Acids on Inhibiting HP Growth

Various concentrations of test compounds were used to examine their effect on the growth of HP 43504 and HP 238. The aqueous solutions of tested compounds used in this experiment comprised 4-phenylbutyrate (4-PB), 2-phenylbutyrate (2-PB), butyrate, gama-aminobutyric acid (GABA), 2-aminobutyric acid (2-AB),2, 4-diaminobutyric acid (2,4-DAB) and sodium valproate (Valproate). HP 43504 and the clinically isolated strain HP 238 were used for testing their susceptibility to various compounds in disc diffusion assays. After 2 days' pre-culture in CDC agar plates (Becton Dickinson, Cockeysville, Md., USA), HP 43504 or HP 238 was suspended in a Brucella broth (BBL, Cockeysville, Md., USA) containing 5% sucrose to adjust its clone density at 1×10⁸CFU/ml. Then 100 μl of the bacterial suspension was inoculated evenly onto a CDC agar (Becton Dickinson, Cockeysville, Md., USA) or Brucella agar plate (supplemented with 10% horse serum) to allow growth to form bacterial lawns. The paper discs, 8 mm in diameter, (from Toyo Roshi Kaisha) containing 40 μl of each test compound were applied to the agar plates. The agar plates were later transferred to a microaerobic jar to incubate at 37° C. The diameter (mm) of each inhibition zone (disc diffusion zone) that indicates zones of bacterium non-growth was measured 48 hours later, as indicated by the number for each compound in Table 1. The results are shown in Table 1.

TABLE 1
Disc diffusion assay against HP for various compounds

| | | Disc diffusion diameter (mm) | | | |
|---|---|---|---|---|---|
| | | HP 43504 | | HP 238 | |
| | | CDC | | CDC | |
| Compounds | mg/ml | agar | Brucella agar | agar | Brucella agar |
| 4-PB | 100 | nd | 45 | Nd | 64 |
| 4-PB | 50 | 17 | 33 | 23 | 40 |
| 4-PB | 25 | 8 | 8 | 9 | 26 |
| 4-PB | 5 | 8 | 8 | 8 | 8 |
| 2-PB | 50 | 8 | 18 | 14 | 24 |
| Butyrate | 50 | 8 | nd | 11 | nd |
| GABA | 100 | 8 | 8 | 8 | 8 |
| 2-AB | 100 | 8 | 8 | 8 | 8 |
| 2,4-DAB | 100 | 8 | 8 | 8 | 8 |
| Valproate | 16 | 8 | nd | 8 | nd |
| Valproate | 8 | 8 | nd | 8 | nd |
| Valproate | 4 | 8 | nd | 8 | nd |
| Valproate | 2 | 8 | nd | 8 | nd |

The median PB dose recommended for ornithine transcarbamylase-deficient patients is 352 mg/kg/day, and no side effects related to therapy were observed. Therefore, we chose biologically available and safe concentrations of 5 to 100 mg/ml for 4-PB and related compounds in the present experiments. The results indicate that 4-PB and 2-PB, compared to other compounds, have much larger disc diffusion diameters in both HP 43504 and HP 238 lawns, indicating that they have much better anti-HP activity. The paper discs treated with 4-PB solutions of 25 to 100 mg/ml showed anti-HP activity. Therefore, a gradient of concentration that is below 25 to 50 mg/ml might be still effective in inhibiting HP growth. In considering biological availability, for example, when a 60-kg subject takes 21,120 mg (352 mg/kg x 60 kg) of 4-PB in 100 ml distilled water by two divided fractions, the expected local concentration in the gastric lumen would be 50 to 200 mg/ml, which would be enough to inhibit HP proliferation based on the present findings.

Butyric acid is also a member of the HDAC inhibitors, and is classified as the short-chain fatty acid class, and was shown to have a bactericidal effect on HP. Sodium butyrate was used in the present experiment. However, its disc diffusion capacity was much smaller than 4-PB or 2-PB for both HP strains in the present studies.

The three other short-chain fatty acids (GABA, 2-AB, 2,4-DAB) that have a similar structural backbone to that of butyrate, 4-PB and 2-PB have no obvious anti-HP activity, since there is no clear zone outside the paper disc even at a higher concentration (100 mg/ml). Therefore, anti-HP activity does not seem to be a general phenomenon for short-chain fatty acids with butyric backbone.

Valproate, an HDAC inhibitor, is the drug of choice for primary generalized epilepsy and partial seizures. However, Valproate has dose-related side effects including nausea, vomiting, tremor, sedation, confusion or irritability, hair loss or curling of hair, endocrine effects (insulin resistance, anovulatory cycles, amenorrhea, and polycystic ovary syndrome) and weight gain. Patients with an underlying urea cycle disorder may suffer from fatal encephalopathy from acute hyperammonemia. The most serious idiosyncratic effect is hepatotoxicity, mainly in patients younger than 2 years old and with polytherapy. Therefore, much consideration is required when prescribing Valproate, and its usual recommended dose is 20˜40 mg/kg/day. The tested biologically available and safe concentrations for Valproate are therefore chosen from 2 mg/ml to 16 mg/ml. The data showed that Valproate shows no effect on inhibiting the growth of both HP strains outside the disc. It therefore has no obvious anti-HP activity with biologically available and safe concentrations. Therefore, anti-HP activity does not seem to be a general phenomenon for HDAC inhibitors.

### EXAMPLE 3

HP Susceptibility Assays for PB in Combination with Various Antibiotics

HP 43504 and HP 2378 were further used for testing PB in combination with various antibiotics in disc diffusion assays. After 2 days' pre-culture in a microaerophilic condition (O₂ 5%, CO₂ 10%, N₂ 85%) at 37° C., HP was suspended in a Brucella broth containing 5% sucrose to adjust its clone density at 1×10⁸ CFU/ml. Then 100 μl of the bacterial suspension was inoculated onto a CDC agar plate. The paper discs (from BBL), 8 mm in diameter, containing 40 μl of each test combination was applied to the agar plates. The agar plates were later transferred to a microaerobic jar to incubate at 37° C. The diameter of each inhibition zone (clear zone size) in the bacterial lawn was measured 48 hours later. The tested combinations used in this experiment comprised 4-PB (50 mg/ml) with amoxicillin (AMO), metronidazole (MTZ) or tetracycline (TC). The clinically recommended doses for these antibiotics are Amoxicillin 40 mg/kg/day, Metronidazole 15 to 20 mg/kg/day, and Tetracycline 25 to 50 mg/kg/day. The concentration for each test antibiotic was chosen based on the therapeutic window against HP microorganism and clinically biological availability and safety, comprising AMO 0.015 μg/20 μl, MTZ 5 μg/ 20 μl, and TC 0.5 μg/ 20 μl. The results are shown in Table 2.

TABLE 2
Disc diffusion assay against HP for PB plus various antibiotics

| Compounds | concentrations | HP 43504 | HP 238 |
|---|---|---|---|
| AMO + water | 0.015 μg/20 μl + 20 μl | 8 mm | 8 mm |
| AMO + 4-PB | 0.015 μg/20 μl + 2 mg/20 μl | 18 | 22 |
| MTZ + water | 5 μg/20 μl + 20 μl | 8 | 16 |
| MTZ + 4-PB | 5 μg/20 μl + 2 mg/20 μl | 48 | 22 |
| TC + water | 0.5 μg/20 μl + 20 μl | 8 | 15 |
| TC + 4-PB | 0.5 μg/20 μl + 2 mg/20 μl | 16 | 20 |

When AMO was applied at a low concentration (0.015 μg/ 20 μl of AMO for 20 μl) that cannot create an obvious clear zone on both HP lawns (8 mm disc diffusion diameter for both types of lawns), the addition of 4-PB (2 mg/ 20 μl of 4-PB for 20 μl) can light up the resolution zones. The same phenomenon was observed for combinations of 4-PB with MTZ or TC. Therefore, short-chain fatty acid like 4-PB or 2-PB is a good alternative for the therapy of HP infection, whether administered alone or in combination with traditional anti-HP antibiotics or other regimens.

Thus, a pharmaceutical composition that can eradicate HP infection locally, decrease the toxicity and resistance of the anti-HP regimens, and promote healing of gastrointestinal ulcer and inhibit the late sequela of HP infection (tumor cell proliferation) may increase the therapeutic gain in treating HP infection and/or an associated disease.

All of the features disclosed in this specification may be combined in any combination. Each feature disclosed in this specification may be replaced by an alternative feature serving the same, equivalent or similar purpose. Thus, unless expressly stated otherwise, each feature disclosed is only an example of a generic series of equivalent or similar features.

From the above description, one skilled in the art can easily ascertain the essential characteristics of the present invention and without departing from the spirit and scope thereof can make various changes and modifications of the invention to adapt it to various usages and conditions. For example, compounds structurally analogous to short-chain fatty acids described above can also be used to practice the present invention. Thus, other embodiments are also within the claims.

The invention may be varied in many ways by a person skilled in the art. Such variations are not to be regarded as a departure from the spirit and scope of the invention, and all such modifications are intended to be included within the scope of the following claims.

What is claimed is:

1. A method for treating a Helicobacter pylori (HP) infection and/or an associated disease in an individual, comprising administering to the individual a therapeutically effective amount of a pharmaceutical composition containing a short-chain fatty acid or a pharmaceutically acceptable salt thereof, alone or in combination with one or more anti-HP agents, and a pharmaceutically acceptable carrier,
wherein the short-chain fatty acid has the formula: 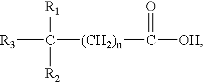
in which
R₁ and R₂, independently, are C₁-C₈alkyl or H,
R₃ is aryl or heteroaryl, and
n is 0,1,2,3,4,5 or 6.

2. The method according to claim 1, wherein R₃ is aryl.

3. The method according to claim 2, wherein R₃ is phenyl.

4. The method according to claim 1, wherein n is 0.

5. The method according to claim 1, wherein R₃ is phenyl.

6. The method according to claim 5, wherein one of R₁ and R₂ is H, and the other is ethyl.

7. The method according to claim 1, wherein n is 1.

8. The method according to claim 7, wherein R₃ is phenyl.

9. The method according to claim 8, wherein one of R₁ and R₂ is H, and the other is methyl.

10. The method according to claim 1, wherein n is 2.

11. The method according to claim 10, wherein R₃ is phenyl.

12. The method according to claim 11, wherein each of R₁ and R₂ is H.

13. The method according to claim 1, wherein the associated disease is selected from the group consisting of chronic atrophic gastritis, gastric ulcer, duodenal ulcer, gastric adenocarcinoma, gastroesophageal reflux, peptic esophagitis, squamous cell esophageal cancer, mucosa-associated lymphoid tissue lymphomas, iron deficiency anemia, skin disease, coronary artery disease, idiopathic thrombocytopenic purpura and rheumatological diseases.

14. The method according to claim 1, wherein the anti-HP agents in combination with the short-chain fatty acid is selected from the group consisting of omeprazole, lansoprazole, amoxicillin, tetracycline, doxycycline, minocycline, metronidazole, tinidazole, clarithromycin, roxithromycin and bismuth subsalicylate.

15. The method according to claim 1, wherein the pharmaceutical composition is administered orally.

16. The method according to claim 15, wherein the pharmaceutical composition is administered in the form of soft or hard capsules, tablets, powders, granules, solutions, suspensions or the like.

17. The method according to claim 1, wherein the pharmaceutical composition is administered intravenously or intra-arterially.

18. The method according to claim 17, wherein the pharmaceutical composition is administered in the form of an injection solution, a drip infusion formulation, or a liposome formulation.

19. The method according to claim 1, wherein the therapeutically effective amount of the short-chain fatty acid is in the range of from 20 mg/kg/day to 500 mg/kg/day.
